(19) United States
(12) Patent Application Publication
(10) Pub. No.: US 2008/0191163 A1
(43) Pub. Date: Aug. 14, 2008

(54) Laser-Assisted Etching Using Gas Compositions Comprising Unsaturated Fluorocarbons

(76) Inventor: Michael T. Mocella, Chadds Ford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/025,980

(22) Filed: Feb. 5, 2008

Related U.S. Application Data
(60) Provisional application No. 60/900,504, filed on Feb. 9, 2007.

(51) Int. Cl. C09K 13/00; C07C 19/08

(52) U.S. Cl. 252/79.1; 570/175

(57) ABSTRACT

Disclosed herein are laser cutting/etching assist fluids and methods of use thereof. The compounds include unsaturated fluorocarbons appropriate for use in laser assist applications.

### FIELD OF THE INVENTION

The disclosure herein relates to assist gases for laser etching processes. The disclosure herein further relates to use of unsaturated fluorocarbons as assist gases for laser processing of metal objects, metal oxide objects, and silicon objects such as semiconductor wafers, silicon nitride parts, and silicon carbide parts, as well as silicon containing glasses.

### BACKGROUND OF THE INVENTION

Numerous methods of using lasers to etch, micromachine or cut metal- and silicon-containing objects and films are known. Lasers used in these processes include CO2, Nb:YAG, excimer and other sources. The substrates used in these processes include, for example, silicon and its oxides, carbides and nitrides, and metals such as titanium, vanadium, chromium, manganese, zirconium, niobium, molybdenum, tantalum, and tungsten, and their compounds with elements such as carbon, oxygen, and nitrogen.

Assist gases are used in many of these processes to improve the cutting speed, etching rate, or regularity or quality of the kerf or microstructure produced in process. For example, inert gases such as argon may be used to carry ablated material away from the processing region, particularly in processes where thicker materials are being cut or etched, or in semiconductor processing where particulate ablated material may be produced. The flow of these gases may further aid the process by cooling the object in the area surrounding the processing region, i.e. the heat affected zone (HAZ). Reactive assist gases may also be used to improve the laser processing method. For example, halogen-containing gases may be used to react with ablated material to change the material to gaseous species, thereby preventing them from redepositing. In addition, these reactive assist gases may be used to form reactive species, either through thermal decomposition at the kerf surface or by direct absorption of beam energy by the gas, where the reactive species then chemically etches the object. These reactive gases include fluorine containing gases such as SF6, NF3, CF4 and COF2.

However, these gases have a variety of limitations in practice, including toxicity and environmental impact, making their handling and use in commercial settings economically disadvantaged. Moreover there may be a trade-off between the rate of etching (or cutting) and the stability of the assist gas. The greater the stability of the assist gas, the more energy input is necessary to generate reactive species, which in turn leads to a greater HAZ. The lower the stability of the reactive gas, the greater the expense of material handling and disposal.

Thus, there is a need in this field for novel assist gases. Such substitutes should have a low ozone depletion potential (ODP) and a low global warming potential (GWP). In addition, the assist gas should be reactive enough to decompose at or below the ablation temperature of the substrates to be cut or etched, should be highly volatile or gaseous, and leave no residue following their use. At the same time, such gases should also be low in toxicity, not form flammable mixtures in air, and have acceptable thermal and chemical stability for storage and transportation. Finally, such assist gases should have sufficient stability such that they do efficiently remove heat from the HAZ without decomposing.

### SUMMARY OF THE INVENTION

There is provided according to the present disclosure assist gases which have low ODP, GWP, are comparatively non-toxic, and meet the reactivity/stability ratio for economical use in the semiconductor industry and related technologies for thin film processing such as etching silicon containing glasses used in displays.

One aspect of the invention is an assist gas composition including a fluorine source that has at least one fluorocarbon or hydrofluorocarbon selected from the group consisting of:
(i) a hydrofluorocarbon having the formula E- or Z-R¹CH═CHR², wherein R¹ and R² are, independently, C₁ to C₆ perfluoroalkyl groups; and
(ii) a fluorocarbon or hydrofluorocarbon selected from the group consisting of CF₃CH═CF₂, CHF₂CF═CF₂, CF₃CF═CHF, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCHCF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHCF₃, CF₃CF₂CH═CH₂, CF₂═CHCF₂CF₃, CF₂═CFCHFCF₃, CF₂═CFCF₂CHF₂, CHF₂CH═CHCF₃, (CF₃)₂C═CHCF₃, CF₃CF═CHCF₂CF₃, CF₃CH═CFCF₂CF₃, CF₃CF═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CH F═C(CF₃)(CH₃), CH₂═C(CHF₂)₂, CF₃CF═CFCH₃, CH₃CF═CHCF₃, CF₂═CFCF₂CF₂CF₃, CHF═CFCF₂CF₂CF₃, CF₂═CHCF₂CF₂CF₃, CF₂═CFCF₂CF₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CFCF₂CF₂CF₃, CHF═CFCF₂CF₂CHF₂, CH₂═C(CF₃)CF₂CF₃, CF₂═CHCH(CF₃)₂, CHF═CHCF(CF₃)₂, CF₂═C(CF₃)CH₂CF₃, CH₂═CFCF₂CF₂CHF₂, CF₂═CHCF₂CH₂CF₃, CF₃CF═C(CF₃)(CH₃), CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂C(CF₃)CH₂CF₃, (CF₃)₂C═CHC₂F₅, (CF₃)₂CFCF═CHCF₃, CH₂═CHC(CF₃)₃, (CF₃)₂C═C(CH₃)(CF₃), CH₂═CFCF₂CH(CF₃)₂, CF₃CF═C(CH₃)CF₂CF₃, CF₃CH═CHCH(CF₃)₂, CH₂═CHCF₂CF₂CF₂CHF₂, (CF₃)₂C═CHCF₂CH₃, CH₂═C(CF₃)CH₂C₂F₅, CH₂═CHCH₂CF₂C₂F₅, CH₂═CHCH₂CF₂C₂F₅, CF₃CF₂CF═CFC₂H₅, CH₂═CHCH₂CF(CF₃)₂, CF₃CF═CHCH(CF₃)(CH₃), (CF₃)₂C═CFC₂H5, cyclo-CF₂CF₂CF₂CH═CH—, cyclo-CF₂CF₂CH═CH—, CF₃CF₂CF₂C(CH₃)═CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂, CF₃CF₂CF₂CF₂CH═CH₂, CF₃CH═CHCF₂CF₃, CF₃CF₂CH═CHCF₂CF₃, CF₃CH═CHCF₂CF₂CF₃, CF₃CF═CFC₂F₅, CF₃CF═CFCF₂CF₂C₂F₅, CF₃CF₂CF═CFCF₂C₂F₅, CF₃CH═CFCF₂CF₂C₂F₅, CF₃CF═CHCF₂CF₂C₂F₅, CF₃CF₂CH═CFCF₂C₂F₅, CF₃CF₂CF═CHCF₂C₂F₅, C₂F₅CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHCF₂CF₃, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrCF₂CF₃, CHBr═CHCF₂CF₃, CH₂═CHCF₂CF₂Br, CH₂═CHCBrFCF₃, CH₃CBr═CHCF₃, CF₃CBr═CHCH₃, (CF₃)₂C═CHBr, CF₃CF═C BrCF₂CF₃, E-CHF₂CBr═CFC₂F₅, Z-CHF₂CBr═CFC₂F₅, CF₂═CBrCHFC₂F₅, (CF₃)₂CFCBr═CH₂, CHBr═CF(CF₂)₂CHF₂, CH₂═CBrCF₂C₂F₅, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, CF₃CF₂CF₂CBr═CH₂, and CF₃(CF₂)₃CBr═CH₂.

A further aspect provides for a method of cutting or etching an object using a laser beam, including the steps of
providing an object to be cut or etched,
providing an atmosphere containing an assist gas at the surface of the object, wherein the assist gas has a fluorine source that is
(i) a hydrofluorocarbon having the formula E- or Z-R¹CH═CHR², wherein R¹ and R² are, independently, C₁ to C₆ perfluoroalkyl groups; and
(ii) a fluorocarbon or hydrofluorocarbon selected from the group consisting of CF₃CH═CF₂, CHF₂CF═CF₂, CF₃CF═CHF, CHF₂CH═CHF, CF₃CF═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCHCF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHCF₃, CF₃CF₂CH═CH₂, CF₂═CHCF₂CF₃, CF₂═CFCHFCF₃, CF₂═CFCF₂CHF₂, CHF₂CH═CHCF₃, (CF₃)₂C═CHCF₃, CF₃CF═CHCF₂CF₃, CF₃CH═CFCF₂CF₃, CF₃CF═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CHF═C(CF₃)(CH₃), CH₂═C(CHF₂)₂, CF₃CF═CFCH₃, CH₃CF═CHCF₃, CF₂═CFCF₂CF₂CF₃, CHF═CFCF₂CF₂CF₃, CF₂═CHCF₂CF₂CF₃, CF₂═CFCF₂CF₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CFCF₂CF₂CF₃, CHF═CFCF₂CF₂CHF₂, CH₂═C(CF₃)CF₂CF₃, CF₂═CHCH(CF₃)₂, CHF═CHCF(CF₃)₂, CF₂═C(CF₃)CH₂CF₃, CH₂═CFCF₂CF₂CHF₂, CF₂═CHCF₂CH₂CF₃, CF₃CF═C(CF₃)(CH₃), CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂C(CF₃)CH₂CF₃, (CF₃)₂C═CHC₂F₅, (CF₃)₂CFCF═CHCF₃, CH₂═CHC(CF₃)₃, (CF₃)₂C═C(CH₃)(CF₃), CH₂═CFCF₂CH(CF₃)₂, CF₃CF═C(CH₃)CF₂CF₃, CF₃CH═CHCH(CF₃)₂, CH₂═CHCF₂CF₂CF₂CHF₂, (CF₃)₂C═CHCF₂CH₃, CH₂═C(CF₃)CH₂C₂F₅, CH₂═CHCH₂CF₂C₂F₅, CH₂═CHCH₂CF₂C₂F₅, CF₃CF₂CF═CFC₂H₅, CH₂═CHCH₂CF(CF₃)₂, CF₃CF═CHCH(CF₃)(CH₃), (CF₃)₂C═CFC₂H5, cyclo-CF₂CF₂CF₂CH═CH—, cyclo-CF₂CF₂CH═CH—, CF₃CF₂CF₂C(CH₃)═CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂, CF₃CF₂CF₂CF₂CH═CH₂, CF₃CH═CHCF₂CF₃, CF₃CF₂CH═CHCF₂CF₃, CF₃CH═CHCF₂CF₂CF₃, CF₃CF═CFC₂F₅, CF₃CF═CFCF₂CF₂C₂F₅, CF₃CF₂CF═CFCF₂C₂F₅, CF₃CH═CFCF₂CF₂C₂F₅, CF₃CF═CHCF₂CF₂C₂F₅, CF₃CF₂CH═CFCF₂C₂F₅, CF₃CF₂CF═CHCF₂C₂F₅, C₂F₅CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHCF₂CF₃, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrCF₂CF₃, CHBr═CHCF₂CF₃, CH₂═CHCF₂CF₂Br, CH₂═CHCBrFCF₃, CH₃CBr═CHCF₃, CF₃CBr═CHCH₃, (CF₃)₂C═CHBr, CF₃CF═CBrCF₂CF₃, E-CHF₂CBr═CFC₂F₅, Z-CHF₂CBr═CFC₂F₅, CF₂═CBrCHFC₂F₅, (CF₃)₂CFCBr═CH₂, CHBr═CF(CF₂)₂CHF₂, CH₂═CBrCF₂C₂F₅, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, CF₃CF₂CF₂CBr═CH₂, or CF₃(CF₂)₃CBr═CH₂;
directing a laser beam onto the surface of the work piece at the location to be cut or etched such that the material of the object at the location is removed.

The removal of the material takes place by either ablation of the material followed by reaction with the assist gas to form gaseous species, by the generation of reactive species from the assist gas which chemically etch the surface of the film but do not substantially etch the HAZ, or, preferably, both by ablation and chemical etching of the film at the kerf. Other objects and advantages will become apparent to those skilled in the art upon reference to the detailed description that hereinafter follows.

### DETAILED DESCRIPTION OF THE INVENTION

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

One aspect provides compounds having the formula E- or Z-R¹CH═CHR² (Formula I), wherein R¹ and R² are, independently, C₁ to C₆ perfluoroalkyl groups. Examples of R¹ and R² groups include, but are not limited to, CF₃, C₂F₅, CF₂CF₂CF₃, CF(CF₃)₂, CF₂CF₂CF₂CF₃, CF(CF₃)CF₂CF₃, CF₂CF(CF₃)₂, C(CF₃)₃, CF₂CF₂CF₂CF₂CF₃, CF₂CF₂CF(CF₃)₂, C(CF₃)₂C₂F₅, CF₂CF₂CF₂CF₂CF₂CF₃, CF(CF₃) CF₂CF₂C₂F₅, and C(CF₃)₂CF₂C₂F₅. Exemplary, non-limiting Formula I compounds are presented in Table 1.

TABLE 1

| Code | Structure | Chemical Name |
|---|---|---|
| F11E | CF₃CH═CHCF₃ | 1,1,1,4,4,4-hexafluorobut-2-ene |
| F12E | CF₃CH═CHC₂F₅ | 1,1,1,4,4,5,5,5-octafluoropent-2-ene |
| F13E | CF₃CH═CHCF₂C₂F₅ | 1,1,1,4,4,5,5,6,6,6-decafluorohex-2-ene |
| F13iE | CF₃CH═CHCF(CF₃)₂ | 1,1,1,4,5,5,5-heptafluoro-4-(trifluoromethyl)pent-2- |
| | | ene |
| F22E | C₂F₅CH═CHC₂F₅ | 1,1,1,2,2,5,5,6,6,6-decafluorohex-3-ene |
| F14E | CF₃CH═CH(CF₂)₃CF₃ | 1,1,1,4,4,5,5,6,6,7,7,7-dodecafluorohept-2-ene |
| F14iE | CF₃CH═CHCF₂CF—(CF₃)₂ | 1,1,1,4,4,5,6,6,6-nonafluoro-5-(trifluoromethyl)hex- |
| | | 2-ene |
| F14sE | CF₃CH═CHCF(CF₃)—C₂F₅ | 1,1,1,4,5,5,6,6,6-nonafluoro-4-(trifluoromethyl)hex- |
| | | 2-ene |
| F14tE | CF₃CH═CHC(CF₃)₃ | 1,1,1,5,5,5-hexafluoro-4,4-bis(trifluoromethyl)pent- |
| | | 2-ene |
| F23E | C₂F₅CH═CHCF₂C₂F₅ | 1,1,1,2,2,5,5,6,6,7,7,7-dodecafluorohept-3-ene |
| F23iE | C₂F₅CH═CHCF(CF₃)₂ | 1,1,1,2,2,5,6,6,6-nonafluoro-5-(trifluoromethyl)hex- |
| | | 3-ene |
| F15E | CF₃CH═CH(CF₂)₄CF₃ | 1,1,1,4,4,5,5,6,6,7,7,8,8,8-tetradecafluorooct-2-ene |
| F15iE | CF₃CH═CH—CF₂CF₂CF(CF₃)₂ | 1,1,1,4,4,5,5,6,7,7,7-undecafluoro-6- |
| | | (trifluoromethyl)hept-2-ene |
| F15tE | CF₃CH═CH—C(CF₃)₂C₂F₅ | 1,1,1,5,5,6,6,6-octafluoro-4,4- |
| | | bis(trifluoromethyl)hex-2-ene |
| F24E | C₂F₅CH═CH(CF₂)₃CF₃ | 1,1,1,2,2,5,5,6,6,7,7,8,8,8-tetradecafluorooct-3-ene |
| F24iE | C₂F₅CH═CHCF₂CF—(CF₃)₂ | 1,1,1,2,2,5,5,6,7,7,7-undecafluoro-6- |
| | | (trifluoromethyl)hept-3-ene |
| F24sE | C₂F₅CH═CHCF(CF₃)—C₂F₅ | 1,1,1,2,2,5,6,6,7,7,7-undecafluoro-5- |
| | | (trifluoromethyl)hept-3-ene |
| F24tE | C₂F₅CH═CHC(CF₃)₃ | 1,1,1,2,2,6,6,6-octafluoro-5,5- |
| | | bis(trifluoromethyl)hex-3-ene |

Compounds of Formula I may be prepared by contacting a perfluoroalkyl iodide of the formula R¹I with a perfluoroalkyltrihydroolefin of the formula R²CH═CH₂ to form a trihydroiodoperfluoroalkane of the formula R¹CH₂CHIR². This trihydroiodoperfluoroalkane can then be dehydroiodinated to form R¹CH═CHR². Alternatively, the olefin R¹CH═CHR² may be prepared by dehydroiodination of a trihydroiodoperfluoroalkane of the formula R¹CHICH₂R² formed in turn by reacting a perfluoroalkyl iodide of the formula R²¹ with a perfluoroalkyltrihydroolefin of the formula R¹CH═CH₂.

Said contacting of a perfluoroalkyl iodide with a perfluoroalkyltrihydroolefin may take place in batch mode by combining the reactants in a suitable reaction vessel capable of operating under the autogenous pressure of the reactants and products at reaction temperature. Suitable reaction vessels include those fabricated from stainless steels, in particular of the austenitic type, and the well-known high nickel alloys such as Monel® nickel-copper alloys, Hastelloy® nickel based alloys and Inconel® nickel-chromium alloys. Alternatively, the reaction may take be conducted in semi-batch mode in which the perfluoroalkyltrihydroolefin reactant is added to the perfluoroalkyl iodide reactant by means of a suitable addition apparatus such as a pump at the reaction temperature.

The ratio of perfluoroalkyl iodide to perfluoroalkyltrihydroolefin should be between about 1:1 to about 4:1, preferably from about 1.5:1 to 2.5:1. Ratios less than 1.5:1 tend to result in large amounts of the 2:1 adduct as reported by Jeanneaux, et. al. in Journal of Fluorine Chemistry, Vol. 4, pages 261-270 (1974).

Temperatures for contacting of said perfluoroalkyl iodide with said perfluoroalkyltrihydroolefin are preferably within the range of about 150° C. to 300° C., more preferably from about 170° C. to about 250° C., and most preferably from about 180° C. to about 230° C. Pressures for contacting of said perfluoroalkyl iodide with said perfluoroalkyltrihydroolefin are preferably the autogenous pressure of the reactants at the reaction temperature.

Suitable contact times for the reaction of the perfluoroalkyl iodide with the perfluoroalkyltrihydroolefin are from about 0.5 hour to 18 hours, preferably from about 4 to about 12 hours.

The trihydroiodoperfluoroalkane prepared by reaction of the perfluoroalkyl iodide with the perfluoroalkyltrihydroolefin may be used directly in the dehydroiodination step or may preferably be recovered and purified by distillation prior to the dehydroiodination step.

In yet another embodiment, the contacting of a perfluoroalkyliodide with a perfluoroalkyltrihydroolefin takes place in the presence of a catalyst.

In one embodiment, a suitable catalyst is a Group VIII transition metal complex. Representative Group VIII transition metal complexes include, without limitation, zero valent NiL₄ complexes, wherein the ligand, L, can be a phosphine ligand, a phosphite ligand, a carbonyl ligand, an isonitrile ligand, an alkene ligand, or a combination thereof. In one such embodiment, the Ni(0)L₄ complex is a NiL₂(CO)₂ complex. In one particular embodiment, the Group VIII transition metal complex is bis(triphenyl phospine)nickel(0) dicarbonyl. In one embodiment, the ratio of perfluoroalkyl iodide to perfluoroalkyltrihydroolefin is between about 3:1 to about 8:1. In one embodiment, the temperature for contacting of said perfluoroalkyl iodide with said perfluoroalkyltrihydroolefin in the presence of a catalyst, is within the range of about 80° C. to about 130° C. In another embodiment, the temperature is from about 90° C. to about 120° C.

In one embodiment, the contact time for the reaction of the perfluoroalkyl iodide with the perfluoroalkyltrihydroolefin in the presence of a catalyst is from about 0.5 hour to about 18 hours. In another embodiment, the contact time is from about 4 to about 12 hours.

The dehydroiodination step is carried out by contacting the trihydroiodoperfluoroalkane with a basic substance. Suitable basic substances include alkali metal hydroxides (e.g., sodium hydroxide or potassium hydroxide), alkali metal oxide (for example, sodium oxide), alkaline earth metal hydroxides (e.g., calcium hydroxide), alkaline earth metal oxides (e.g., calcium oxide), alkali metal alkoxides (e.g., sodium methoxide or sodium ethoxide), aqueous ammonia, sodium amide, or mixtures of basic substances such as soda lime. Preferred basic substances are sodium hydroxide and potassium hydroxide.

Said contacting of the trihydroiodoperfluoroalkane with a basic substance may take place in the liquid phase preferably in the presence of a solvent capable of dissolving at least a portion of both reactants. Solvents suitable for the dehydroiodination step include one or more polar organic solvents such as alcohols (e.g., methanol, ethanol, n-propanol, isopropanol, n-butanol, isobutanol, and tertiary butanol), nitriles (e.g., acetonitrile, propionitrile, butyronitrile, benzonitrile, or adiponitrile), dimethyl sulfoxide, N,N-dimethylformamide, N,N-dimethylacetamide, or sulfolane. The choice of solvent depends on the solubility of the basic substance, the solubility of the perfluoroalkyl iodide, and the solubility of the perfluoroalkyltrihydroolefin as well as the boiling point of the product, and the ease of separation of traces of the solvent from the product during purification. Typically, ethanol or isopropanol are good solvents for the reaction. Separation of solvent from the product may be effected by distillation, extraction, phase separation, or a combination of the three.

Typically, the dehydroiodination reaction may be carried out by addition of one of the reactants (either the basic substance or the trihydroiodoperfluoroalkane) to the other reactant in a suitable reaction vessel. Said reaction vessel may be fabricated from glass, ceramic, or metal and is preferably agitated with an impellor or other stirring mechanism.

Temperatures suitable for the dehydroiodination reaction are from about 10° C. to about 100° C., preferably from about 20° C. to about 70° C. The dehydroiodination reaction may be carried out at ambient pressure or at reduced or elevated pressure. Of note are dehydroiodination reactions in which the compound of Formula I is distilled out of the reaction vessel as it is formed.

Alternatively, the dehydroiodination reaction may be conducted by contacting an aqueous solution of said basic substance with a solution of the trihydroiodoperfluoroalkane in one or more organic solvents of lower polarity such as an alkane (e.g., hexane, heptane, or octane), aromatic hydrocarbon (e.g., toluene), halogenated hydrocarbon (e.g., methylene chloride, ethylene dichloride, chloroform, carbon tetrachloride, or perchloroethylene), or ether (e.g., diethyl ether, methyl tert-butyl ether, tetrahydrofuran, 2-methyl tetrahydrofuran, dioxane, dimethoxyethane, diglyme, or tetraglyme) in the presence of a phase transfer catalyst. Suitable phase transfer catalysts include quaternary ammonium halides (e.g., tetrabutylammonium bromide, tetrabutylammonium hydrosulfate, triethylbenzylammonium chloride, dodecyltrimethylammonium chloride, and tricaprylylmethylammonium chloride), quaternary phosphonium halides (e.g., triphenylmethylphosphonium bromide and tetraphenylphosphonium chloride), cyclic ether compounds known in the art as crown ethers (e.g., 18-crown-6 and 15-crown-5).

Alternatively, the dehydroiodination reaction may be conducted in the absence of solvent by adding the trihydroiodoperfluoroalkane to one or more solid or liquid basic substance(s).

Suitable reaction times for the dehydroiodination reactions are from about 15 minutes to about six hours or more depending on the solubility of the reactants. Typically the dehydroiodination reaction is rapid and requires about 30 minutes to about three hours for completion.

The compound of formula I may be recovered from the dehydroiodination reaction mixture by phase separation, optionally after addition of water, by distillation, or by a combination thereof.

The compositions of the present disclosure may comprise a single compound of Formula I, for example, one of the compounds in Table 1, or may comprise a combination of compounds of Formula I.

The compositions of the present disclosure may comprise a single compound as listed, for example, in Table 1, or may comprise a combination of compounds from Table 1. Additionally, many of the compounds in Table 1 may exist as different configurational isomers or stereoisomers. The present disclosure is intended to include all single configurational isomers, single stereoisomers, or any combination thereof. For instance, F11 E (CF₃CH═CHCF₃) is meant to represent the E-isomer, Z-isomer, or any combination or mixture of both isomers in any ratio. Another example is F24E (C₂F₅CH═CH(n-C₄F₉)) by which is represented the E-isomer, Z-isomer, or any combination or mixture of both isomers in any ratio.

Global warming potentials (GWPs) are an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100 year time horizon is commonly the value referenced.

The present invention will provide compositions that have zero or low ozone depletion potential and low global warming potential (GWP). The fluoroolefins of the present invention or mixtures of fluoroolefins of this invention with other assist gas compositions will have global warming potentials that are less than many fluorine-containing assist gas compositions currently in use. Typically, the fluoroolefins of the present invention are expected to have GWP of less than about 25. One aspect of the present invention is to provide an agent with a global warming potential of less than 1000, less than 500, less than 150, less than 100, or less than 50. Another aspect of the present invention is to reduce the net GWP of assist gas compositions by adding fluoroolefins to said agents.

The present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

The compositions of the present disclosure may be prepared by any convenient method to combine the desired amounts of the individual components. A preferred method is to weigh the desired component amounts and thereafter combine the components in an appropriate vessel. Agitation may be used, if desired.

In a preferred embodiment, compounds of the present disclosure are useful in laser processing applications using assist gases. In addition to the inventive compounds described above, compounds presented in Table 2 can be used in assist gas applications.

TABLE 2

| Code | Structure | IUPAC Name |
|---|---|---|
| HFC-1225ye | CF3CF═CHF | 1,2,3,3,3-pentafluoro-1-propene |
| HFC-1225yc | CHF2CF═CF2 | 1,1,2,3,3-pentafluoro-1-propene |
| HFC-1234ye | CHF2CF═CHF | 1,2,3,3-tetrafluoro-1-propene |
| HFC-1234yf | CF3CF═CH2 | 2,3,3,3-tetrafluoro-1-propene |
| HFC-t-1234ze | CF3CH═CHF | 1,3,3,3-tetrafluoro-1-propene |
| HFC-1234yc | CH2FCF═CF2 | 1,1,2,3-tetrafluoro-1-propene |
| HFC-1234zc | CHF2CH═CF2 | 1,1,3,3-tetrafluoro-1-propene |
| HFC-1234ye | CHF2CF═CHF | 1,2,3,3-tetrafluoro-1-propene |
| HFC-1243s | C3H3F3 | |
| HFC-1243yf | CHF2CF═CH2 | 2,3,3-trifluoro-1-propene |
| HFC-1243zf | CF3CH═CH2 | 3,3,3-trifluoro-1-propene |
| HFC-1243yc | CH3CF═CF2 | 1,1,2-trifluoro-1-propene |
| HFC-1243zc | CH2FCH═CF2 | 1,1,3-trifluoro-1-propene |
| HFC-1243ye | CHF2CF═CHF | 1,2,3-trifluoro-1-propene |
| HFC-1243ze | CHF2CH═CHF | 1,3,3-trifluoro-1-propene |
| FC-1318my | CF₃CF═CFCF₃ | 1,1,1,2,3,4,4,4-octafluorobut-2-ene |
| FC-1318cy | CF₂═CFCF₂CF₃ | 1,1,2,3,3,4,4,4-octafluorobut-1-ene |
| HFC-1327my | CF₃CF═CHCF₃ | 1,1,1,2,4,4,4-heptafluorobut-2-ene |
| HFC-1327cz | CF₂═CHCF₂CF₃ | 1,1,3,3,4,4,4-heptafluorobut-1-ene |
| HFC-1327ye | CHF═CFC₂F₅ | 1,2,3,3,4,4,4-heptafluoro-1-butene |
| HFC-1327py | CHF₂CF═CFCF₃ | 1,1,1,2,3,4,4-heptafluoro-2-butene |
| HFC-1327cye | CF₂═CFCHFCF3 | 1,1,2,3,4,4,4-heptafluorobut-1-ene |
| HFC-1327cyc | CF₂═CFCF₂CHF₂ | 1,1,2,3,3,4,4-heptafluorobut-1-ene |
| HFC-1327ey | CHF═CFCF₂CF₃ | 1,2,3,3,4,4,4-heptafluorobut-1-ene |
| HFC-1327ct | CF₂═C(CHF₂)CF₃ | 2-(difluoromethyl)-1,1,3,3,3- |
| | | pentafluoroprop-1-ene |
| HFC-1327et | CHF═C(CF₃)₂ | 1,3,3,3-tetrafluoro-2- |
| | | (trifluoromethyl)prop-1-ene |
| HFC-1336fy | CF₃CF₂CF═CH₂ | 2,3,3,4,4,5,5,5-octafluoropent-1-ene |
| HFC-1336qc | CF₂═CFCF2CH₂F | 1,1,2,3,3,4-hexafluorobut-1-ene |
| HFC-1336qy | CH₂FCF═CFCF₃ | 1,1,1,2,3,4-hexafluorobut-2-ene |
| HFC-1336ze | CHF═CHCF₂CF₃ | 1,3,3,4,4,4-hexafluorobut-1-ene |
| HFC-1336pz | CHF₂CH═CFCF₃ | 1,1,1,2,4,4-hexafluorobut-2-ene |
| HFC-1336pe | CHF₂CHFCF═CF₂ | 1,1,2,3,4,4-hexafluorobut-1-ene |
| HFC-1336eye | CHF═CFCHFCF₃ | 1,2,3,4,4,4-hexafluorobut-1-ene |
| HFC-1336ze | CHF═CHCF₂CF₃ | 1,3,3,4,4,4-hexafluoro-1-butene |
| HFC-1336pyy | CHF₂CF═CFCHF₂ | 1,1,2,3,4,4-hexafluoro-2-butene |
| HFC-1336mzy | CHF₂CF═CHCF₃ | 1,1,1,3,4,4-hexafluorobut-2-ene |
| HFC-1336czc | CHF₂CF₂CH═CF₂ | 1,1,3,3,4,4-hexafluorobut-1-ene |
| HFC-1336eyc | CHF═CFCF₂CHF₂ | 1,2,3,3,4,4-hexafluorobut-1-ene |
| HFC-1336cyf | CF₂═CFCH₂CF₃ | 1,1,2,4,4,4-hexafluorobut-1-ene |
| HFC-1336cze | CF₂═CHCHFCF₃ | 1,1,3,4,4,4-hexafluorobut-1-ene |
| HFC-1336ft | CH₂═C(CF₃)₂ | 3,3,3-trifluoro-2-(trifluoromethyl)prop- |
| | | 1-ene |
| HFC-1429mzt | (CF₃)₂C═CHCF₃ | 1,1,1,4,4,4-hexafluoro-2- |
| | | (trifluoromethyl)but-2-ene |
| HFC-1429eyy | CHF═CFCF(CF₃)₂ | 1,2,3,4,4,4-hexafluoro-3- |
| | | (trifluoromethyl)but-1-ene |
| HFC-1429cyz | CF₂═CFCH(CF₃)₂ | 1,1,2,4,4,4-hexafluoro-3- |
| | | (trifluoromethyl)but-1-ene |
| HFC-1429czy | CF₂═CHCF(CF₃)₂ | 1,1,3,4,4,4-hexafluoro-3- |
| | | (trifluoromethyl)but-1-ene |
| HFC-1429myz | CF₃CF═CHCF₂CF₃ | 1,1,1,2,4,4,5,5,5-nonafluoropent-2- |
| | | ene |
| HFC-1429mzy | CF₃CH═CFCF₂CF₃ | 1,1,1,3,4,4,5,5,5-nonafluoropent-2- |
| | | ene |
| HFC-1429eyc | CHF═CFCF₂CF₂CF₃ | 1,2,3,3,4,4,5,5,5-nonafluoropent-1- |
| | | ene |
| HFC-1429czc | CF₂═CHCF₂CF₂CF₃ | 1,1,3,3,4,4,5,5,5-nonafluoropent-1- |
| | | ene |
| HFC-1429cycc | CF₂═CFCF₂CF₂CHF₂ | 1,1,2,3,3,4,4,5,5-nonafluoropent-1- |
| | | ene |
| HFC-1429pyy | CHF₂CF═CFCF₂CF₃ | 1,1,2,3,4,4,5,5,5-nonafluoropent-2- |
| | | ene |
| HFC-1429myyc | CF₃CF═CFCF₂CHF₂ | 1,1,1,2,3,4,4,5,5-nonafluoropent-2- |
| | | ene |
| HFC-1429myye | CF₃CF═CFCHFCF₃ | 1,1,1,2,3,4,5,5,5-nonafluoropent-2- |
| | | ene |
| HFC-1438ezcc | CHF═CHCF₂CF₂CF₃ | 1,3,3,4,4,5,5,5-octafluoropent-1-ene |
| HFC-1438etme | CHF═C(CF₃)CHFCF₃ | 1,3,4,4,4-pentafluoro-2- |
| | | (trifluoromethyl)but-1-ene |
| HFC-1438ftmc | CH₂═C(CF₃)CF₂CF₃ | 3,3,4,4,4-pentafluoro-2- |
| | | (trifluoromethyl)but-1-ene |
| HFC-1438czz | CF₂═CHCH(CF₃)₂ | 1,1,4,4,4-pentafluoro-4- |
| | | (trifluoromethyl)but-1-ene |
| HFC-1438ezy | CHF═CHCF(CF₃)₂ | 1,3,4,4,4-pentafluoro-4- |
| | | (trifluoromethyl)but-1-ene |
| HFC-1438ctmf | CF₂═C(CF₃)CH₂CF₃ | 1,1,4,4,4-pentafluoro-2- |
| | | (trifluoromethyl)but-1-ene |
| PFBE (HFC- | CF₃CF₂CF₂CF₂CH═CH₂ | 3,3,4,4,5,5,6,6,6-nonafluorohex-1-ene |
| 1549fzcc) | | |
| HFC-1549czcf | CF₂═CHCF₂CH₂CF₂CF₃ | 1,1,3,3,5,5,6,6,6-nonafluorohex-1-ene |
| HFC-1549myzf | CF₃CF═CHCH₂CF₂CF₃ | 1,1,1,2,5,5,6,6,6-nonafluorohex-2- |
| | | ene |
| HFC-1549fzt | CH₂═CHC(CF₃)₃ | 4,4,4-trifluoro-3,3- |
| | | bis(trifluoromethyl)but-1-ene |
| HFC- | (CF₃)₂C═C(CH₃)CF₃ | 1,1,1,4,4,4-hexafluoro-1- |
| 1549mmttm | | (trifluoromethyl)but-2-ene |
| HFC- | CF₂═C(CF₃)CH₂CHFCF₃ | 1,1,4,5,5,5-hexafluoro-2- |
| 1549ctmfe | | (trifluoromethyl)but-1-ene |
| HFC-1549ctsc | CF₂═C(CH₃)CF₂CF₂CF₃ | 1,1,3,3,4,4,5,5,5-nonafluoro-2- |
| | | methylpent1-ene |
| HFC-1549etsf | CHF═C(CF₃)CH₂CF₂CF₃ | 1,4,4,5,5,5-hexafluoro-2- |
| | | (trifluoromethyl)pent-1-ene |
| HFC-1549fzym | CH₂═CHCF(CF₃)CF₂CF₃ | 3,4,4,5,5,5-hexafluoro-3- |
| | | (trifluoromethyl)pent-1-ene |
| HFC-1549fycz | CH₂═CFCF₂CH(CF₃)₂ | 2,3,3,5,5,5-hexafluoro-4- |
| | | (trifluoromethyl)pent-1-ene |
| HFC-1549mytp | CF₃CF═C(CH₃)CF₂CF₃ | 1,1,1,2,4,4,5,5,5-nonafluoro-3- |
| | | methylpent-2-ene |
| HFC-1549mzzz | CF₃CH═CHCH(CF₃)₂ | 1,1,1,5,5,5-hexafluoro-4- |
| | | (trifluoromethyl)pent-2-ene |
| FC-141-10myy | CF₃CF═CFC₂F₅ | 1,1,1,2,3,4,4,5,5,5-decafluoro-2- |
| | | pentene |
| HFC-152- | (CF₃)₂CFCF═CHCF₃ | 1,1,1,3,4,5,5,5-octafluoro-4- |
| 11mmyyz | | (trifluoromethyl)pent-2-ene |
| HFC-152- | CF₃)₂C═CHC₂F₅ | 1,1,1,4,4,5,5,5-octafluoro-2- |
| 11mmtz | | (trifluoromethyl)-2-pentene |
| HFC-151- | CF₃CF═CFCF₂CF₂CF₃ | 1,1,1,2,3,4,4,5,5,6,6,6- |
| 12myyc | | dodecafluorohex-2-ene |
| HFC-151- | CF₂═C(CF₃)CF₂CF₂CF₃ | 1,1,3,3,4,4,5,5,5-nonafluoro-2- |
| 12ctmc | | (trifluoromethyl)pent-1-ene |
| HFC-151- | CF₂═CFCF₂CF(CF₃)₂ | 1,1,2,3,3,4,5,5,5-nonafluoro-4- |
| 12cycym | | (trifluoromethyl)pent-1-ene |
| HFC-151- | CF₃CF═CFCF(CF₃)₂ | 1,1,1,2,3,4,5,5,5--nonafluoro-4- |
| 12cyyym | | (trifluoromethyl)pent-2-ene |
| HFC-151- | CF₃CF═C(CF₃)CF₂CF₃ | 1,1,1,2,4,4,5,5,5-nonafluoro-3- |
| 12mytm | | (trifluoromethyl)pent-2-ene |
| HFC-151- | (CF₃)₂C═CFCF₂CF₃ | 1,1,1,3,4,4,5,5,5-nonafluoro-1- |
| 12mmty | | (trifluoromethyl)pent-1-ene |
| HFC-151- | CF₂═CFC(CF₃)₃ | 1,1,2,4,4,4-hexafluoro-3,3- |
| 12cytmm | | bis(trifluoromethyl)but-1-ene |
| HFC-151- | CF₂═C(CF₃)CF(CF₃)₂ | 1,1,3,4,4,4-hexafluoro-2,3- |
| 12ctmym | | bis(trifluoromethyl)but-1-ene |
| HFC-151- | CF₃(CF₂)₃CF═CF₂ | 1,1,2,3,3,4,4,5,5,6,6,6- |
| 12cycc | | dodecafluorohex-1-ene |
| HFC-151- | CF₃CF₂CF═CFCF₂CF₃ | 1,1,1,2,2,3,4,5,5,6,6,6- |
| 12mcy | | dodecafluorohex-3-ene |
| HFC-151- | (CF₃)₂C═C(CF₃)₂ | 1,1,1,4,4,4-hexafluoro-2,3- |
| 12mmt | | bis(trifluoromethyl)but-2-ene |
| HFC-153- | CF₂═CHCF₂CF₂CF₂CF₂H | 1,1,3,3,4,4,5,5,6,6-decafluorohex-1- |
| 10czccc | | ene |
| HFC-153- | CHF═CFCF₂CF₂CF₂CF₂H | 1,2,3,3,4,4,5,5,6,6-decafluorohex-1- |
| 10eyccc | | ene |
| HFC-153- | CF₃CH═CFCF₂CF₂CF₂H | 1,1,1,3,4,4,5,5,6,6-decafluorohex-2- |
| 10mzycc | | ene |
| HFC-153- | CF₂═C(CF₃)CH₂CF₂CF₃ | 1,1,4,4,5,5,5-heptafluoro-2- |
| 10ctmf | | (trifluoromethyl)pent-1-ene |
| HFC-153- | (CF₃)₂C═CFCH₂CF₃ | 1,1,1,3,5,5,5-heptafluoro-2- |
| 10mmtyc | | (trifluoromethyl)pent-2-ene |
| HFC-153- | CF₃CH═CFCH(CF₃)₂ | 1,1,1,3,5,5,5-heptafluoro-4- |
| 10mzyz | | (trifluoromethyl)pent-2-ene |
| FC-C-1316cc | Cyclo-CF₂CF₂CF═CF— | hexafluorocyclobutene |
| FC-C-1418y | Cyclo-CF₂CF═CFCF₂CF₂— | octafluorocyclopentene |
| FC-C-151-10y | Cyclo-CF₂CF═CFCF₂CF₂CF₂ | decafluorocyclohexane |

The compounds listed in Table 2 are available commercially or may be prepared by processes known in the art or as described herein.

1,1,1,4,4,4-hexafluoro-2-butene (CF₃CH═CHCF₃) may be prepared from 1,1,1,4,4,4-hexafluoro-2-iodobutane (CF₃CHICH₂CF₃) by reaction with KOH using a phase transfer catalyst at about 60° C. The synthesis of 1,1,1,4,4,4-hexafluoro-2-iodobutane may be carried out by reaction of perfluoromethyl iodide (CF₃I) and 3,3,3-trifluoropropene (CF₃CH═CH₂) at about 200° C. under autogenous pressure for about 8 hours.

1,1,1,2,3,4-hexafluoro-2-butene (CF₃CF═CFCH₂F) may be prepared by dehydrofluorination of 1,1,1,2,3,3,4-heptafluorobutane (CH₂FCF₂CHFCF₃) using solid KOH.

1,1,1,2,4,4-hexafluoro-2-butene (CF₃CF═CHCHF₂) may be prepared by dehydrofluorination of 1,1,1,2,2,4,4-heptafluorobutane (CHF₂CH₂CF₂CF₃) using solid KOH.

1,1,1,3,4,4-hexafluoro-2-butene (CF₃CH═CFCHF₂) may be prepared by dehydrofluorination of 1,1,1,3,3,4,4-heptafluorobutane (CF₃CH₂CF₂CHF₂) using solid KOH.

Compositions of the present disclosure can comprise a single compound as listed, for example, in Table 2, or may comprise a combination of compounds from Table 2 or, alternatively, a combination of compounds from Table 2 and Formula I.

Additionally, many of the compounds in Table 2 may exist as different configurational isomers or stereoisomers. When the specific isomer is not designated, the present disclosure is intended to include all single configurational isomers, single stereoisomers, or any combination thereof. For instance, 1,1,1,2,4,4,5,5,5-nonafluoropent-2-ene is meant to represent the E-isomer, Z-isomer, or any combination or mixture of both isomers in any ratio. Another example is HFC-1336pz, by which is represented the E-isomer, Z-isomer, or any combination or mixture of both isomers in any ratio.

The present assist gases are fluids, and may be liquids or gases under ambient conditions, but are preferably utilized for the present laser assist applications in a gaseous state. Where a liquid fluid is used, it is preferably vaporized by exposure of the compound to the kerf or by absorption of energy directly from the laser beam. In the alternative, the volatility of the liquid may be such that its vapor pressure is such that the partial pressure of the compound over the liquid may be used.

The term “kerf” is usually used to denote the notch or cut made in a object by a cutting tool, in this instance, a laser.

Lasers useful for the practice of the invention may include those which emit light in ultraviolet, visible and infrared ranges, preferably in the range of 150-1500 nm, more preferably 193-1152 nm.

Laser can be operated in pulse or continuous mode, and include CO₂ lasers, Nb:YAG lasers, excimer lasers, ArF lasers, KrF lasers, HeNe lasers, ruby lasers and the like, depending on the absorbtion and thermal characteristics of the object to be processed and the assist gas selected.

Objects to be etch/ablated/removed include silicon-containing objects, such as silicon wafers in the semiconductor industry, and its compounds with oxygen, nitrogen, and or carbon, including silicon oxide silicon nitride, silicon oxynitride, silicon carbide, silicon carbonitride, and silicon containing glasses used in the display industry, as well as various materials known as organosilicate glasses. Objects also include metals, including titanium, vanadium, chromium, manganese, zirconium, niobium, molybdenum, tantalum, and tungsten—and their compounds with silicon, oxygen, and or nitrogen, including metal silicides, oxides and nitrides.

The process can be conducted at atmospheric pressure or in partial vacuum. In practice, in addition to the composition of the invention, the gas may included added gases such as O2 or another oxygen source such as the various oxides of nitrogen, carbon and/or sulfur, as well as water vapor. Other fluorine sources such as NF3, SF6, and CF4 (or other CxFy perfluorinated compounds with x<6). Flow rates of gases may range from 0.01 to 10 slm.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit, and scope of the present disclosure. More specifically, it will be apparent that certain agents which are chemically related may be substituted for the agents described herein while the same or similar results would be achieved. All such similar substitutes and modifications apparent to those skilled in the art are deemed to be within the spirit, scope, and concept of the present disclosure as defined by the appended claims.

### EXAMPLES

The present disclosure is further defined in the following Examples. It should be understood that these Examples, while indicating preferred embodiments, are given by way of illustration only. From the above discussion and these Examples, one skilled in the art can ascertain the preferred features, and without departing from the spirit and scope thereof, can make various changes and modifications to adapt it to various uses and conditions.

### Example 1

### Synthesis of 1,1,1,4,4,5,5,6,6,7,7,7-dodecafluorohept-2-ene (F14E)

### Synthesis of C₄F₉CH₂CHICF₃

Perfluoro-n-butyliodide (180.1 gm, 0.52 moles) and 3,3,3-trifluoropropene (25.0 gm, 0.26 moles) were added to a 400 ml Hastelloy™ shaker tube and heated to 200° C. for 8 hours under autogenous pressure, which increased to a maximum of 428 PSI. The product was collected at room temperature. The above reaction was carried out again at these conditions and the products combined. It was then repeated doubling the amount of perfluoro-n-butyliodide and 3,3,3-trifluoropropene in the same 400 ml reactor. In this case the pressure increased to 573 PSI. The products of the three reactions were combined and distilled to give 322.4 gm of C₄F₉CH₂CHICF₃ (52.20/35 mm) in 70% yield.

### Conversion of C₄F₉CH₂CHICF₃ to F14E

C₄F₉CH₂CHICF₃ (322.4 gm, 0.73 moles) was added dropwise via addition funnel to a 2 L round bottom flask equipped with stir a bar and connected to a packed distillation column and still head. The flask contained isopropyl alcohol (95 ml), KOH (303.7 gm, 0.54 moles) and water (303 ml). Product was collected, washed with sodium metabisulfite, water, dried with MgSO₄ and distilled through a 6″ column filled with glass helices. The product, F14E (173.4 gm, 76%) boils at 78.2° C. It was characterized by ¹⁹F NMR (δ-66.7 (CF₃, m, 3F), −81.7 (CF₃, m 3F), −124.8 (CF₂, m, 2F), −126.4 (CF₂, m, 2F), and −114.9 ppm (CF₂, m, 2F)) ¹H NMR (δ 6.4\tilde{5}) in chloroform-d solution.

### Example 2

### Synthesis of 1,1,1,2,2,5,5,6,6,7,7,8,8,8-tetradecafluorooct-3-ene (F24E)

### Synthesis of C₄F₉CHICH₂C₂F₅

Perfluoroethyliodide (220 gm, 0.895 mole) and 3,3,4,4,5,5,6,6,6-nonafluorohex-1-ene (123 gm, 0.50 mole) were added to a 400 ml Hastelloy™ shaker tube and heated to 200° C. for 10 hours under autogenous pressure. The product from this and two others carried out under similar conditions were combined and washed with two 200 mL portions of 10 wt % aqueous sodium bisulfite. The organic phase was dried over calcium chloride and then distilled to give 277.4 gm of C₄F₉CH₂CHICF₃ (79-81° C./67-68 mm Hg) in 37% yield.

### Conversion of C₄F₉CHICH₂C₂F₅ to F24E

A 1 L round bottom flask equipped with a mechanical stirrer, addition funnel, condenser, and thermocouple was charged with C₄F₉CHICH₂C₂F₅ (277.4 gm, 0.56 moles) and isopropanol (217.8 g). The addition funnel was charged with a solution of potassium hydroxide (74.5 g, 1.13 moles) dissolved in 83.8 g of water. The KOH solution was added dropwise to the flask with rapid stirring over the course of about one hour as the temperature slowly increased from 21° C. to 42° C. The reaction mass was diluted with water and the product recovered by phase separation. The product was washed with 50 mL portions of 10 wt % aqueous sodium bisulfite and water, dried over calcium chloride, and then distilled at atmospheric pressure. The product, F24E (128.7 gm, 63%) boils at 95.5° C. It was characterized by ¹⁹F NMR (δ-81.6 (CF₃, m, 3F), −85.4 (CF₃, m 3F), −114.7 (CF₂, m, 2F), −118.1 (CF₂, m, 2F), −124.8 ppm (CF₂, m, 2F), −126.3 ppm (CF₂, m, 2F)) and ¹H NMR (□6.48) in chloroform-d solution.

### Example 3

### Synthesis of CF₃CH═CHCF(CF₃)₂

### Synthesis of CF₃CHICH₂CF(CF₃)₂

(CF₃)₂CFI (265 gm, 0.9 moles) and 3,3,3-trifluoropropene (44.0 gm, 0.45 moles) were added to a 400 ml Hastelloy™ shaker tube and heated to 200° C. for 8 hours under autogenous pressure, which increased to a maximum of 585 psi. The product was collected at room temperature to give 110 gm of (CF₃)₂CFCH₂CHICF₃ (76-77° C./200 mm) in 62% yield.

### Conversion of (CF₃)₂CFCH₂CHICF₃ to F13iE

(CF₃)₂CFCH₂CHICF₃ (109 gm, 0.28 moles) was slowly added dropwise via addition funnel to a 500 ml round bottom flask heated to 42° C. equipped with stir a bar and connected to a short path distillation column and dry ice trap. The flask contained isopropyl alcohol (50 ml), KOH (109 gm, 1.96 moles) and water (109 ml). During the addition, the temperature increased from 42 to 55° C. After refluxing for 30 minutes, the temperature in the flask increased to 62° C. Product was collected, washed with water, dried with MgSO₄ and distilled. The product, F13iE (41 gm, 55%), boils at 48-50° C. and was characterized by ¹⁹F NMR (δ-187.6 (CF, m 1F), −77.1 (CF₃, m 6F), −66.3 (CF₃, m 3F) in chloroform-d solution.

### Example 4

### Synthesis of C4F9CHICH₂C2F5

3,3,4,4,5,5,6,6,6-Nonafluorohex-1-ene (20.5 gm, 0.0833 mole), bis(triphenyl phosphine)nickel(0) dicarbonyl (0.53 g, 0.0008 mole), and perfluoroethyliodide (153.6 gm, 0.625 mole) were added to a 210 ml Hastelloy™ shaker tube and heated at 100° C. for 8 hours under autogenous pressure. Analysis of the product by GC-MS indicated the presence of C4F9CHICH₂C2F5 (64.3 GC area %) and the diadduct (3.3 GC area %); the conversion of 3,3,4,4,5,5,6,6,6-nonafluorohex-1-ene was 80.1%.

### Example 5

An atmosphere containing HFC-1225ye is provided over a silicon wafer by streaming 0.01 to 10 slm through nozzle directed towards to the location on the surface to be cut. An excimer laser is then projected onto the wafer at the location, ablating the surface of the wafer. At the kerf, the HFC-1225 decomposes to produce atomic fluorine and polyatomic fluorine-containing radicals, which then react with the ablated particles/liquids to produce gaseous silicon fluoride. At the same time, unreacted assist gas removes heat from the HAZ, reducing the incidence of microcracks and thermal distortion in the silicon wafers's crystalline matrix.

The foregoing written description is only exemplary of the invention, whose limitations are to be found solely in the following claims.

We claim:

1. A laser processing assist fluid comprising at least one fluorocarbon or hydrofluorocarbon selected from the group consisting of:
(i) a hydrofluorocarbon having the formula E- or Z-R¹CH═CHR², wherein R¹ and R² are, independently, C₁ to C₆ perfluoroalkyl groups; and
(ii) a fluorocarbon or hydrofluorocarbon selected from the group consisting of CF₃CH═CF₂, CHF₂CF═CF₂, CF₃CF═CHF, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCHCF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHCF₃, CF₃CF₂CH═CH₂, CF₂═CHCF₂CF₃, CF₂═CFCHFCF₃, CF₂═CFCF₂CHF₂, CHF₂CH═CHCF₃, (CF₃)₂C═CHCF₃, CF₃CF═CHCF₂CF₃, CF₃CH═CFCF₂CF₃, CF₃CF═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CHF═C(CF₃)(CH₃), CH₂═C(CHF₂)₂, CF₃CF═CFCH₃, CH₃CF═CHCF₃, CF₂═CFCF₂CF₂CF₃, CHF═CFCF₂CF₂CF₃, CF₂═CHCF₂CF₂CF₃, CF₂═CFCF₂CF₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CFCF₂CF₂CF₃, CHF═CFCF₂CF₂CHF₂, CH₂═C(CF₃)CF₂CF₃, CF₂═CHCH(CF₃)₂, CHF═CHCF(CF₃)₂, CF₂═C(CF₃)CH₂CF₃, CH₂═CFCF₂CF₂CHF₂, CF₂═CHCF₂CH₂CF₃, CF₃CF═C(CF₃)(CH₃), CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂C(CF₃)CH₂CF₃, (CF₃)₂C═CHC₂F₅, (CF₃)₂CFCF═CHCF₃, CH₂═CHC(CF₃)₃, (CF₃)₂C═C(CH₃)(CF₃), CH₂═CFCF₂CH(CF₃)₂, CF₃CF═C(CH₃)CF₂CF₃, CF₃CH═CHCH(CF₃)₂, CH₂═CHCF₂CF₂CF₂CHF₂, (CF₃)₂C═CHCF₂CH₃, CH₂═C(CF₃)CH₂C₂F₅, CH₂═CHCH₂CF₂C₂F₅, CH₂═CHCH₂CF₂C₂F₅, CF₃CF₂CF═CFC₂H₅, CH₂═CHCH₂CF(CF₃)₂, CF₃CF═CHCH(CF₃)(CH₃), (CF₃)₂C═CFC₂H5, cyclo-CF₂CF₂CF₂CH═CH—, cyclo-CF₂CF₂CH═CH—, CF₃CF₂CF₂C(CH₃)═CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂, CF₃CF₂CF₂CF₂CH═CH₂, CF₃CH═CHCF₂CF₃, CF₃CF₂CH═CHCF₂CF₃, CF₃CH═CHCF₂CF₂CF₃, CF₃CF═CFC₂F₅, CF₃CF═CFCF₂CF₂C₂F₅, CF₃CF₂CF═CFCF₂C₂F₅, CF₃CH═CFCF₂CF₂C₂F₅, CF₃CF═CHCF₂CF₂C₂F₅, CF₃CF₂CH═CFCF₂C₂F₅, CF₃CF₂CF═CHCF₂C₂F₅, C₂F₅CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHCF₂CF₃, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrCF₂CF₃, CHBr═CHCF₂CF₃, CH₂═CHCF₂CF₂Br, CH₂═CHCBrFCF₃, CH₃CBr═CHCF₃, CF₃CBr═CHCH₃, (CF₃)₂C═CHBr, CF₃CF═CBrCF₂CF₃, E-CHF₂CBr═CFC₂F₅, Z-CHF₂CBr═CFC₂F₅, CF₂═CBrCHFC₂F₅, (CF₃)₂CFCBr═CH₂, CHBr═CF(CF₂)₂CHF₂, CH₂═CBrCF₂C₂F₅, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, CF₃CF₂CF₂CBr═CH₂, and CF₃(CF₂)₃CBr═CH₂.

2. The fluid of claim 1, wherein R¹ and R² are, independently, CF₃, C₂F₅, CF₂CF₂CF₃, CF(CF₃)₂, CF₂CF₂CF₂CF₃, CF(CF₃)CF₂CF₃, CF₂CF(CF₃)₂, C(CF₃)₃, CF₂CF₂CF₂CF₂CF₃, CF₂CF₂CF(CF₃)₂, C(CF₃)₂C₂F₅, CF₂CF₂CF₂CF₂CF₂CF₃, CF(CF₃) CF₂CF₂C₂F₅, or C(CF₃)₂CF₂C₂F₅.

3. The fluid of claim 1, wherein the fluorocarbon or hydrofluorocarbon is selected from the group consisting of E-CF₃CH═CHCF₃, Z-CF₃CH═CHCF₃, E-CF₃CH═CFCF₃, Z-CF₃CH═CFCF₃, E-CF₃CF═CFCF₃, Z-CF₃CF═CFCF₃, E-CF₃CH═CHCF₂CF₃, Z-CF₃CH═CHCF₂CF₃, E-CF₃CF═CHCF₂CF₃, Z-CF₃CF═CHCF₂CF₃, E-CF₃CH═CFCF₂CF₃, Z-CF₃CH═CFCF₂CF₃, E-CF₃CF═CFCF₂CF₃, CF₃CF₂CF═CH₂ or Z-CF₃CF═CFCF₂CF₃.

4. The fluid of claim 1, further comprising an additional assist gas selected from the group consisting essentially of NF3, CF4, or COF2.

5. The fluid of claim 1, wherein the fluid is a gas at ambient temperature.

6. The fluid of claim 1, wherein the hydrofluorocarbons is HFC-1225ye, HFC-1234yf, HFC-1234zf, HFC-1336mzz or HFC-1448mzz.

7. The fluid of claim 1, where an oxygen-containing compound is added as part of the composition.

8. The fluid of claim 7, where the oxygen-containing gas includes O2, ozone, oxides of carbon, nitrogen, and sulfur, and water vapor.
